(19) United States
(12) Patent Application Publication
(10) Pub. No.: US 2006/0247527 A1
(43) Pub. Date: Nov. 2, 2006

(54) Ultrasonic diagnostic apparatus, measuring result display apparatus and method of displaying measuring result

(75) Inventor: Toshie Maruyama, Tochigi-ken (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 11/412,082

(22) Filed: Apr. 27, 2006

(30) Foreign Application Priority Data
Apr. 28, 2005 (JP) 2005-133622

(51) Int. Cl. A61B 8/00

(52) U.S. Cl. 600/443

(57) ABSTRACT

An ultrasonic diagnostic apparatus, which obtains a diagnostic image by scanning a fetus in a mother's body with an ultrasonic wave, including a measurement unit configured to obtain from the diagnostic image fetal measuring results according to a plurality of items; a graph processing unit coupled to the measurement unit and configured to generate a graph indicating a relative relation between measuring results and basic values for each of the plurality of items; and a monitor connected to the graph processing unit and configured to display the graph.

### CROSS-REFERENCE TO RELATED APPLICATIONS

This application is based upon and claims the benefit of priority from the prior Japanese Patent Application No. 2005-133622, filed on Apr. 28, 2005, the entire contents of which are incorporated herein by reference.

### BACKGROUND OF THE INVENTION

Measurement of a fetus by an ultrasonic diagnostic apparatus is performed by applying an ultrasonic probe to the mother's body, acquiring the ultrasonic images, and using the fetal measurement function equipped in ultrasonic diagnostic apparatus. There are various aspects to fetal measurement to understand the fetal growth.

The measurement of a fetus is generally performed two or more times. The measurement value obtained each time is recorded on the report area of the patient in the obstetrics department patient database which stores past measuring results. Furthermore, the current measuring result is plotted on a trend graph. A vertical axis of the trend graph indicates measurement values, and a horizontal axis indicates pregnancy week. Mean value and a normal range are also plotted on the trend graph. (For example, see JP09-251364A)

Examples of measured fetal items are:

AA: abdominal area; AC: abdominal circumference; APAD: anteroposterior abdominal diameter; BPD: biparietal diameter; CRL: crown-rump length; EFW: estimated fetal weight; FL: femoral length; FTA: trunk cross-sectional area; GS: gestational sac diameter; HA: head area; HC: head circumference; OFD; occipito frontal diameter; and TAD: transverse abdominal diameter.

In addition, in the case of a fetus having a growth problem (i.e., a high risk fetus), the measurement of other items mentioned below is performed:

CER: cerebellum; Clavicle; Fibula; Foot; Humerus; Kidney; Radius; THD: thoracis diameter; Tibia and Ulna.

In order to show measuring results of these items, trend graphs that respectively correspond to the items are made. The mean value and the normal range is set according to values published by a particular author, or standard values generally known. In Japan, JSUM is generally known as providing standard values.

FIG. 7 exemplifies a measuring result displayed on a monitor of the conventional ultrasonic diagnostic apparatus. In FIG. 7, four kinds of measuring results are illustrated. The upper left measuring result is of the biparietal diameter of fetal head (BPD) displayed on the trend graph in accordance with standard values of Japan (JSUM). The upper right measuring result is of the boundary length of fetal head (HC) displayed on the trend graph in accordance with standard values of Mr. Hadlock as the author. The lower left measuring result is of the anteroposterior trunk diameter (AC) displayed on the trend graph in accordance with standard values of Japan (JSUM). The lower right measuring result is of the femoral length (FL) displayed on the trend graph in accordance with standard values of Japan (JSUM).

Three trend curves are displayed on each of the graphs. A center curve indicates mean value. Moreover, the others indicate a normal range represented as an upper limit curve and a lower limit curve. When a user (i.e., a doctor) estimates the state of fetus growth, the user must comprehensively understand the fetal state. To that end, the user must check many trend graphs of many measurement items. However, because space and viewing ability on a monitor is limited in an ultrasound diagnostic apparatus, it is usual that only about four graphs can be displayed at the same time. For this reason, in order to estimate values of many measurement items, users must switch the display many times and check graphs sequentially. In addition, as is clear from FIG. 7, measuring scales of measurement items sometimes differ from each other. This makes it difficult for users to understand the state of fetal growth in a comprehensive manner.

For judging whether fetus is high-risk fetus or not, it is important to recognize a balance of measuring results about a plurality of measurement items. For example, in the case that the measuring result of one item is larger than a normal range, diagnosing whether the fetus is of high-risk is sometimes different depending on the degree of the overall growth of fetus. When growth of the fetus is fast overall, a fetus having a slightly abnormal larger value of one item may tend not to be considered as high risk. On the other hand, when only one value of an item is too large in comparison with the other items, the fetus generally must be diagnosed as high-risk. However, because the conventional method plots measurement values on trend graph for each measurement item, users could not check many measurement items at one time. Therefore, in the conventional method, diagnosing of a high-risk fetus in a comprehensive manner is difficult.

### BRIEF SUMMARY OF THE INVENTION

According to one aspect of the present invention, there is provided an ultrasonic diagnostic apparatus, measuring result displaying apparatus and measuring result displaying method that enables a user to easily understand fetal state based on a plurality of measurement items in a comprehensive manner.

According to another aspect of the present invention, there is provided an ultrasonic diagnostic apparatus which obtains a diagnostic image by scanning a fetus with an ultrasonic wave, including a measurement unit configured to obtain from the diagnostic image fetal measuring results according to a plurality of items, a graph processing unit coupled to the measurement unit and configured to generate a graph indicating relative relation between measuring results and standard values about each of the plurality of items, and a monitor coupled to the graph processing unit and configured to display the graph.

According to a further aspect of the present invention, there is provided a measuring result display apparatus which displays measuring result of a fetus according to a plurality of items, including a graph processing unit configured to generate a graph indicating a relative relation between measuring results and standard values for each of the plurality of items, and a monitor coupled to the graph processing unit and configured to display the graph.

According to yet another aspect of the present invention, there is provided a method of display fetal measuring results of a plurality of items, generating a graph indicating a relative relation between measuring results based and standard values for each of the plurality of items, and displaying the graph.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a schematic block diagram of a first exemplary embodiment of the ultrasonic diagnostic apparatus of the invention.

FIG. 2 is a flow chart showing flow of fetal measurement in the first exemplary embodiment of the invention.

FIG. 3 is an illustration of a graph order screen displayed on monitor and one example of radar chart made based on the order in the first exemplary embodiment of the invention.

FIG. 4 is an illustration of a graph order screen displayed on a monitor and one example of radar chart made based on the order in a second exemplary embodiment of the invention.

FIG. 5 is an illustration of a one example of a bar chart displayed on a monitor in a third exemplary embodiment of the invention.

FIG. 6 is an illustration of a one example of a line chart displayed on a monitor in a fourth exemplary embodiment of the invention.

FIG. 7 is an illustration of one example of display of fetal measuring results in the conventional ultrasonic diagnostic apparatus.

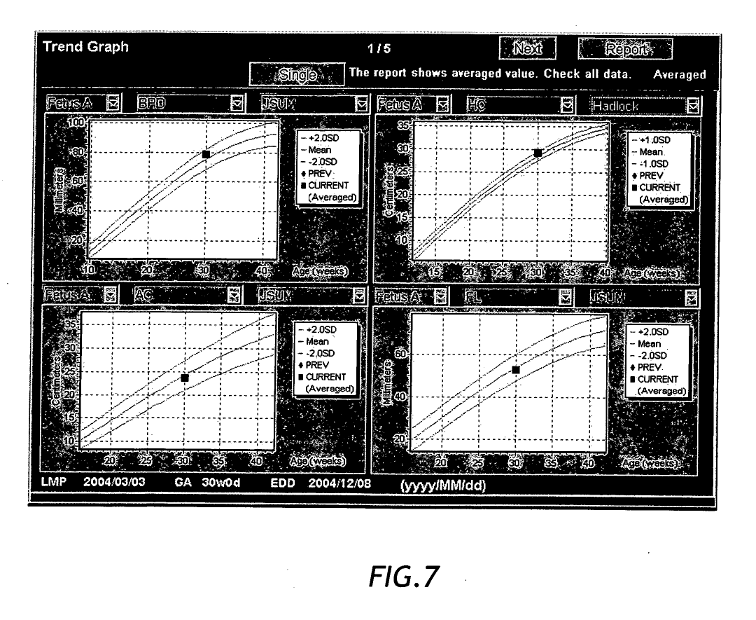

### DETAILED DESCRIPTION OF THE PREFERRED EMBODIMENTS

Referring now to the drawings, wherein like reference numerals designate identical or corresponding parts throughout the several views, various embodiments of the present invention are next described.

### First Exemplary Embodiment

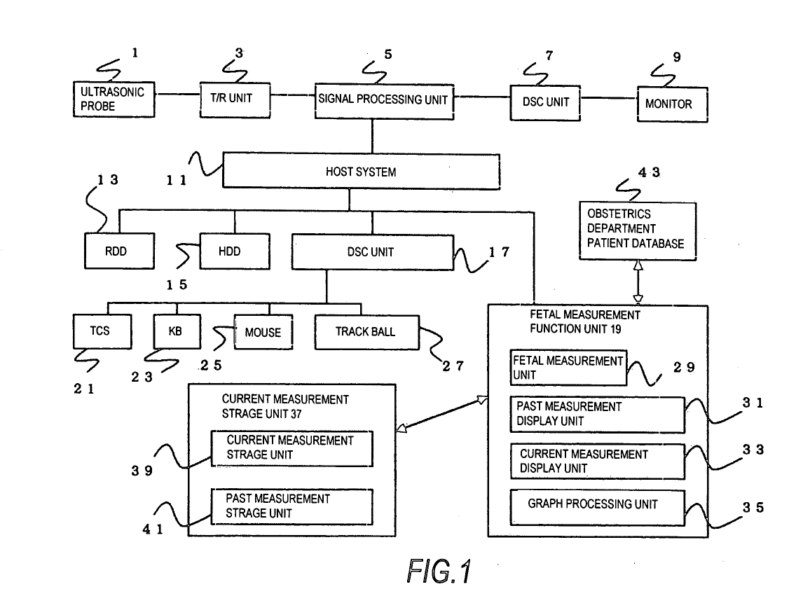

FIG. 1 is a schematic block diagram of ultrasonic diagnostic apparatus in a first exemplary embodiment of the invention. As shown in FIG. 1, the ultrasonic diagnostic apparatus includes an ultrasonic probe 1, transmitting and receiving control unit (T/R unit) 3, signal processing unit 5, digital scan converter unit (DSC unit) 7, monitor 9, host system 11, removable disk drive 13, hard disk drive (HDD) 15, panel sub-system 17 and fetal measurement function unit 19.

Ultrasonic probe 1 controlled by T/R unit 3 transmits ultrasound to a mother's body (not shown in the figure). Ultrasonic probe 1 receives reflected signal from the mother's body and delivers the reflected signal to T/R unit 3.

The T/R unit 3 delivers the reflected signal to the signal processing unit 5, which then generates a B-mode ultrasonic image that represents the mother's body and the fetus. DSC 7 converts the generated B-mode ultrasonic image to a video signal in a TV format. After that, the video signal representing the B-mode ultrasonic image is displayed on the monitor 9.

The panel sub system 17 is connected to touch command screen (TCS) 21, keyboard (KB) 23, mouse 25 and track ball 27. Panel sub-system 17 processes various sorts of input signals among the host system 11 and above mentioned input devices.

The fetal measurement function unit 19 includes fetal measurement unit 29, past measurements display unit 31, current measurement display unit 33 and graph processing unit 35. The fetal measurement unit 19 is connected to item measurements storage unit 37 that includes current measurements storage unit 39 and past measurements storage unit 41. In addition, the fetal measurement function unit 19 is able to access an obstetrics department patient database 43 on a hospital information system (HIS).

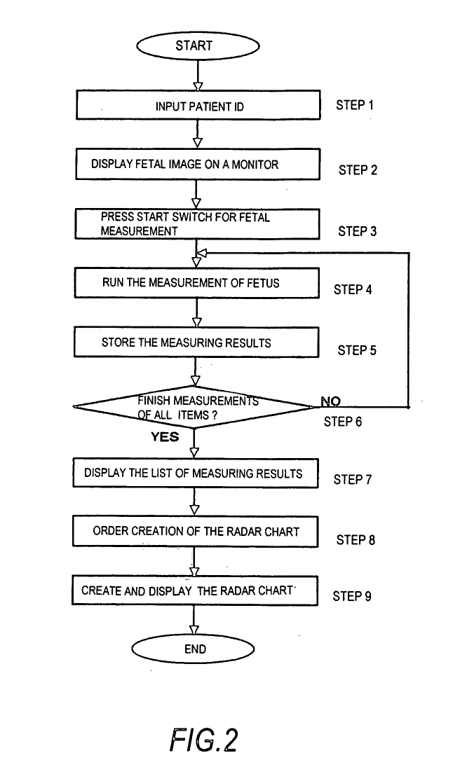

FIG. 2 is a flow chart showing flow of fetal measurement in the first exemplary embodiment of the invention.

For starting fetal measurement, a patient ID is entered at first (step 1). The patient ID identifies the mother. Information of the ID is stored in obstetrics department patient database 43. Next, a user makes a fetal image visible on the monitor 9 by applying ultrasonic probe 1 to the patient (mother's body) (step 2). After acquisition of the fetal image, the user orders fetal measurement to run (step 3). In particular, the user pushes the fetal measurement start switch displayed on TCS 21. This operation signal is delivered to host system 11 via panel sub system 17. Host system 11 makes fetal measurement unit 29 run programmed measurement processes sequentially for a predetermined item (step 4). A measuring result is stored in current measuring result storage unit 37 (step 5).

In step 6, it is judged whether the measurements of all desired items are finished or not. When all of the measurements are finished, processing proceeds to step 7. When a part of the measurements is still not finished, the processing is returned to step 4. In step 7, all of the measuring results are listed on the monitor 9. In other words, current measuring results are read from current measurements storage unit 39 in item measurements storage unit 37, and the measuring results are displayed on the monitor 9.

In this step, because the displayed list is just a row of data of the measuring results, comprehensive analysis of measuring results is difficult. In order to facilitate the analysis, the user orders creation of a radar chart to graph processing unit 35. In this case, for example, the order is transmitted to host system 35 via panel sub system 17 by pushing create radar chart switch, the host system 11 displays a graph order screen according to a preprogrammed procedure, and the graph processing unit 35 creates the radar chart based on the order inputted by use of the screen.

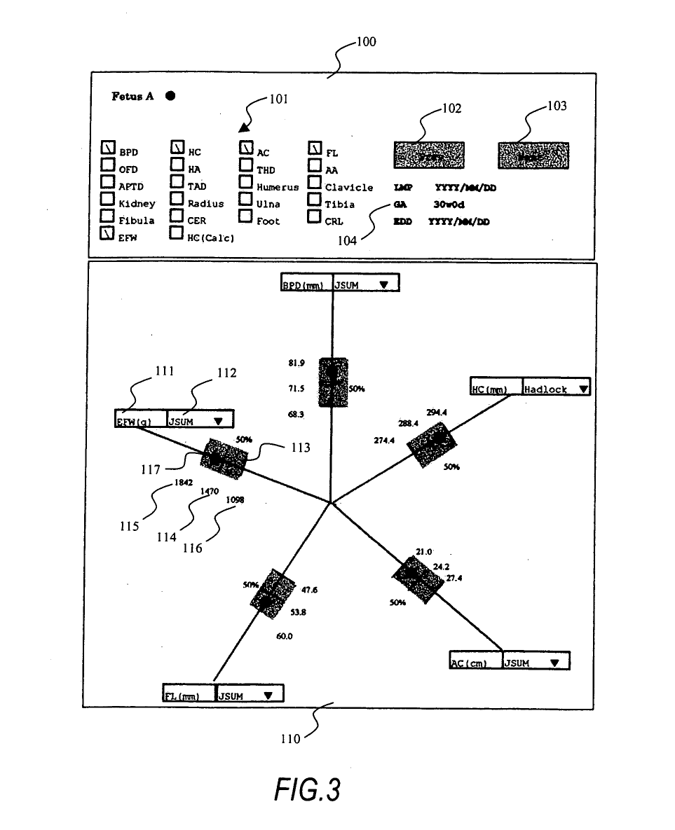

FIG. 3 illustrates a graph order screen 100 displayed on monitor 9 and one example of radar chart 101 made based on the order inputted by use of the screen.

When making of a radar chart is ordered in step 8, measurement items that are usable for a radar chart from the data obtained from Fetus A and check boxes of the items are listed on the upper portion of the monitor 9. Users check each check box that is desirable for display by mouse 25 or the like. FIG. 3 shows a case that the user checked five items (BPD, HC, AC, FL, EFW). In addition, a value of gestational age of the fetus is displayed as (GA) 104 in FIG. 3.

Signals based on this checking are delivered to host system 11 via panel sub system 17, and a radar chart that is created by graph processing unit 35 is displayed on the monitor 9 (step 9). In this example, each axis for each measurement items is percent (%) scaled, and the center of each axis establishes an average value of gestational age (GA) 104. Furthermore, as calibrations of each axis, measurement values can also be displayed. On the center of every axis, a mark of “AVG” or the like that indicate an average value may be displayed.

As average values, upper-limit values or lower-limit values of the measurement items, standard values (JSUM in Japan) are well known, but users may use basic values based on experimental statistical data or published by a dependable author. In consequence, the name of the measurement item 111 and the name of selected basic values 112 as an average value are displayed nearby every axis of the radar chart. The user is able to select the name of the basic values 112 desired as average value by pull-down menu.

As the basic value for every measurement items, for example, JSUM, which provides standard values in Japan, is selected by default. When user changes the name of basic value 112, average value, upper-limit value, lower-limit value, scale of axis and plot position of measurement value is automatically changed in accordance with the selecting. For example, in the radar chart 110 of FIG. 3, a value of author “hadlock” has been selected as the basic value of head circumference (HC), and standard values in Japan (JSUM) is selected as the other basic values.

In the above explanation of this exemplary embodiment, users are able to independently select the basic value in relation to each one of measurement items. However, a pull-down menu for lump selecting of the basic values about all of measurement items may be provided.

In addition, a range 113 between an upper-limit value and a lower limit value may be highlighted by color or hatching. Numeric values of average values 114, numeric values of upper-limit values 115 and numeric values of lower limit values 116 are also displayed, as shown in FIG. 3. Real fetal measurement values are indicated by mark of circle 117 on each axis. In FIG. 3, the axis of EFW has appended thereto a number indicating measurement item 111, name of selected basic values 112, range 113, average values 114, upper-limit values 115 and lower limit values 116. Since the other axes are displayed according to same condition about the other axes reference numeral designations are omitted.

Furthermore, in this exemplary embodiment, the radar chart that indicates a past measuring result may be displayed. As shown in FIG. 3, a “Prev” button 102 and a “Next” button 103 are provided in the graph order screen. For example, when a user click the “Prev” button 102 by the mouse 25, the measuring result of previous time (for example, four weeks ago) is read from the past measurement storage unit 41. Then, in the graph processing unit 35, a radar chart of previous time is made and displayed by condition that is same as the current displayed radar chart. When a user clicks the “Next” button 103, the radar chart of next time is made and displayed.

When a user click the “Prev” button 102 after displaying the radar chart of the previous measuring result yet again, a radar chart of a previous measuring result of all that is made and displayed. It is needless to say that the display of gestational age (GA) is changed according to the operation of the “Prev” button 102 and the “Next” button 103. In addition, a plurality of radar charts that relate to different gestational ages may be made and displayed in a single screen. This makes it an open possibility to check growth status carefully. Similarly, each axis of a radar chart can be displayed with an indication of the “Prev” and/or “Next” value of the measuring result also displayed.

### Second Exemplary Embodiment

As an exemplary embodiment of the invention, the measuring results of a plurality of fetuses are displayed as a radar chart. Since it is well known how to measure a plurality of fetuses by various proposed techniques, the below explanation will be based on the case that measuring results obtained by the technique in fetal measurement function unit 19 are stored in current measuring result storage unit 37.

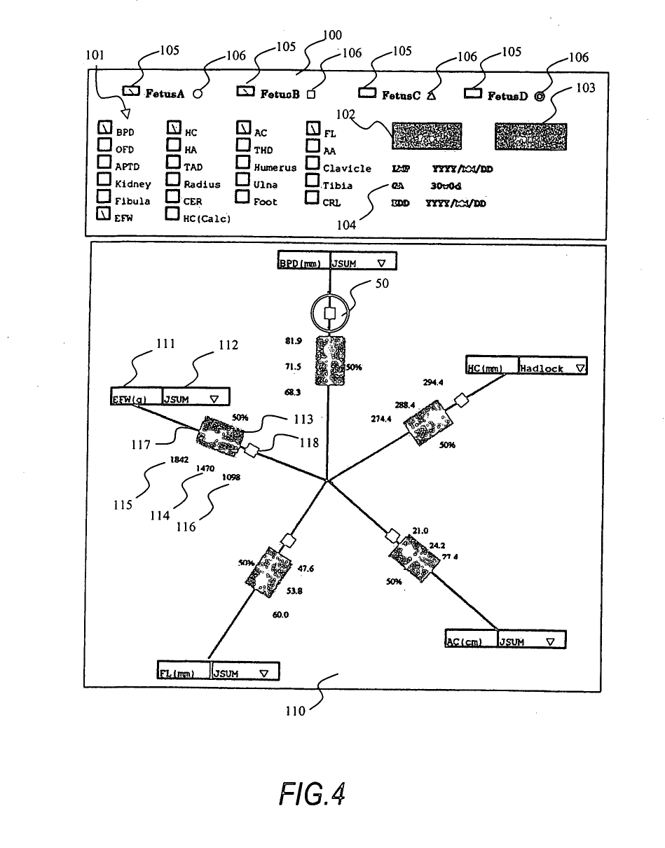

FIG. 4 is a graph order screen 100 displayed on a monitor and one example of radar chart 110 that is made based on the order in the second exemplary embodiment of the invention. In this exemplary embodiment, when a user orders graph processing unit 35 to make a radar chart at step 8 in measurement flow of FIG. 2, a graph order screen, as shown in upper side of FIG. 4, is displayed.

In the graph order screen 100, check boxes 105 for fetus selection is displayed. The check boxes 105 make it possible to select a plurality of fetuses (for example, from Fetus A to Fetus B), whereas, in the case of FIG. 3, only one fetus (Fetus A) is selected. In addition, marks 106 identifying the fetuses are also displayed. This makes it easy to understand the correspondence between measurement value and fetuses. It is needless to say that check boxes 101 for selecting measurement items and the “Next” button 103 or the “Prev” button 102 may be displayed same as the case of FIG. 3.

In the case of FIG. 3, a user has selected Fetus A and Fetus B, and ordered to make radar chart of these. As a measurement items displayed in the radar chart, BPD, HC, AC, FL, EFW have been selected. Thus, on each axis of the radar chart 110, a real measurement value of Fetus A is displayed as a circle mark 117, and a real measurement value of Fetus B is displayed as a square mark 118. This makes it possible to check growth status based on comparison of Fetus A and Fetus B in a comprehensive manner.

### Third Exemplary Embodiment

As a third exemplary embodiment of the invention, measuring results of a fetus is displayed as bar chart. Since it is well known how to measure a fetus by various proposed techniques, below explanation will be based on the case that measuring results obtained by the technique in the fetal measurement function unit 19 are stored in current measuring result storage unit 37.

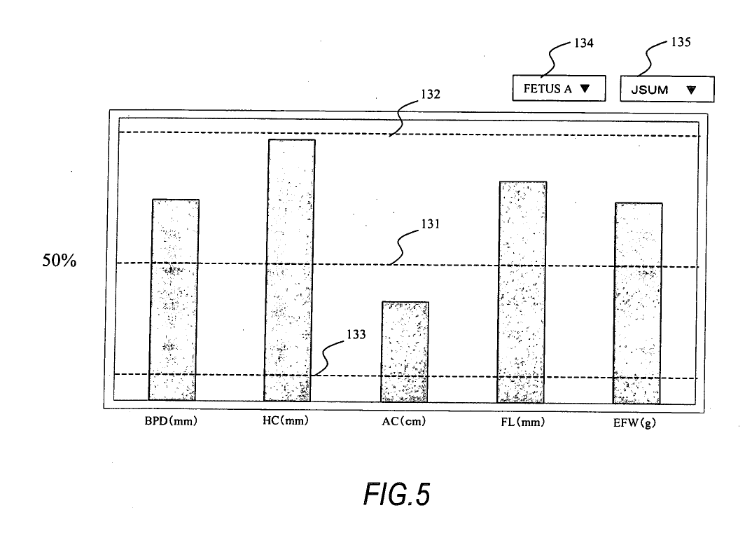

FIG. 5 shows a bar chart of fetus measurement that is displayed on the monitor 9 in the second exemplary embodiment of the invention. In this exemplary embodiment, when a user instructs graph processing unit 35 to make a bar chart at step 8 in measurement flow of FIG. 2, a graph order screen is displayed. Since the graph order screen may be the same as the case of first exemplary embodiment, explanation of that is omitted.

As shown in FIG. 5, the graph processing unit 35 makes a bar chart corresponding to measuring results BPD, HC, AC, FL and EFW of Fetus A. In this chart, a dashed line 131, which is on the center of each longitudinal axis, indicates average values of each measurement items. A dashed line 132, which lies above the dashed line 131, indicates upper limit values about each measurement items. A dashed line 133, which lies below the dashed line 131, indicates lower limit values about each measurement items. For each measurement item, the scale of bar chart is normalized by its average value, upper limit value and lower limit value. Thus, the relative relation between real measurement values and average values, upper limit values and lower limit values is indicated by the displayed bar chart.

In the case of FIG. 5, pull-down menu 135 is displayed. This is for selecting standard values for all measurement items. However, as the case of first exemplary embodiment, configurations for selecting the basic values with respect to each of measurement items may be provided. In addition, in the case of FIG. 5, pull-down menu 134 for selecting a fetus is provided. However, as in the case of the second exemplary embodiment, configurations for displaying measuring results of a plurality of fetus may be provided.

Furthermore, in this exemplary embodiment, the case of displaying a bar chart was explained. However, some applications of well known graphing methods (e.g. line graph, scatter chart) may be provided. In addition, operating configuration for selecting graphing method of measuring result may be provided.

### Fourth Exemplary Embodiment

In a fourth exemplary embodiment of the present invention, measuring results of a fetus are displayed as a time series line graph. Since it is well known how to measure a fetus by various proposed techniques, below explanation will be based on the case that measuring results obtained by the technique in fetal measurement function unit 19 are stored in current measuring result storage unit 37.

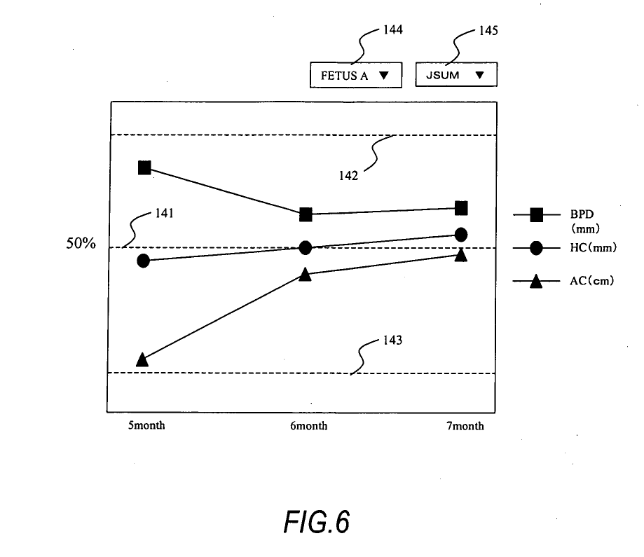

FIG. 6 shows a line graph of fetal measurement that is displayed on the monitor 9 in the fourth exemplary embodiment of the invention. In this exemplary embodiment, when a user instructs graph processing unit 35 to make a line graph at step 8 in the measurement flow of FIG. 2, a graph order screen is displayed. The graph order screen is nearly the same as the case of the first exemplary embodiment. However, in this exemplary embodiment, display for setting a time series range is needed. Thus, for example, an image including a time series range of gestational age is displayed in the graph order screen. The graph processing unit 35, based on this order by the graph order screen, reads measuring results from current measurements storage unit 39 or past measurements storage unit 41. Based on these measuring results, line graphs are created. Since other aspects of the graph order screen may be same as the case of the first exemplary embodiment, explanation thereof is omitted.

As shown in FIG. 6, the graph processing unit 35 makes line graphs corresponding to BPD, HC, and AC of Fetus A. In this chart, a dashed line 141, which is at the center of each vertical axis, indicates average values of every measurement items. A dashed line 142, which lies above the dashed line 141 indicates upper limit values about every measurement item. A dashed line 143, which lies below the dashed line 141, indicates lower limit values about every measurement item. This manner is same as the case of FIG. 5. In each of the measurement items, the vertical scale of each line chart is normalized by the average values, upper limit values and lower limit values. Thus, marks having shapes that correspond to respective measurement items are displayed at points that correspond to relative relation between real measurement values and average values, upper limit values and lower limit values. In this case, square marks indicate HC, and circle marks indicates AC.

In FIG. 6, the horizontal axis indicates gestational age. As shown, a range from fifth month of pregnancy to seventh month of pregnancy is displayed on a monthly basis. Each mark that indicates measurement items is displayed on the location corresponding to each gestational age. In addition, by connecting same kind of marks by lines, the line graph is created.

In the case of FIG. 6, a pull-down menu 145 is displayed. This is for selecting standard values for all measurement items. However, as in the case of first exemplary embodiment, configurations for selecting the standard values with respect to each of measurement items may be provided. In addition, as in the case of second exemplary embodiment, configurations for displaying measuring results of a plurality of fetus may be provided.

Furthermore, in this exemplary embodiment, the case of displaying bar chart was explained. However, some applications of well known graphing methods (e.g. scatter chart) may be provided. In addition, an operating configuration for selecting a graphing method of measuring results may be provided.

According to these exemplary embodiments explained above, measuring results of a plurality of fetal measurement items is displayed. This makes it possible to provide a comprehensive view of measuring result easily. In addition, this makes it possible to estimate balance of the growth state easily. Thus, it is possible to advance throughput and operability.

Furthermore, in the described exemplary embodiments, the charts and the graphs are normalized by standard values. This makes it possible to determine growth state in a comprehensive manner without consciousness of differences of scale. This also decrease the burden on users, makes diagnosing of high-risk fetus easy, and prevents leaks of determination of high-risk fetus

In addition, in the case of FIG. 4, when a diagnosis of high-risk is likely as a result of a big difference between BPD of Fetus B and an average value, in order to alert this, appropriate marks (e.g. line, character) may be displayed on the radar chart, as shown by note 50.

Numerous modifications and variations of the present invention are possible in light of the above teachings. It is therefore to be understood that within the scope of the appended claims, the invention may be practiced otherwise than as specifically described herein.

What is claimed is:

1. An ultrasonic diagnostic apparatus which obtains a diagnostic image by scanning a fetus in a mother's body with an ultrasonic wave, comprising:
a measurement unit configured to obtain from the diagnostic image fetal measuring results according to a plurality of items;
a graph processing unit coupled to the measurement unit and configured to generate a graph indicating a relative relation between measuring results and basic values for each of the plurality of items; and
a monitor connected to the graph processing unit and configured to display the graph.

2. The ultrasonic diagnostic apparatus according to claim 1, wherein
the basic values are average values of measuring results or well known standard values.

3. The ultrasonic diagnostic apparatus according to claim 1, wherein
the standard values are upper-limit values or lower limit values corresponding to a normal range.

4. The ultrasonic diagnostic apparatus according to claim 1, further comprising:
an input unit configured to generate operation information based on input by a user, and to send the operation information to the graph processing unit,
wherein the graph processing unit is configured to create the graph about items selected according to the operation information.

5. The ultrasonic diagnostic apparatus according to claim 1, further comprising:
an input unit configured to generate operation information based on input by a user, and to send operation information to the graph processing unit, and
a measurements storage unit coupled to the measurement unit and configured to store measuring results obtained at a plurality of points of time.
wherein the graph processing unit is coupled to the measurements storage unit and configured to create the graph about measuring result at a point selected from the plurality of points according to the operation information.

6. The ultrasonic diagnostic apparatus according to claim 1, further comprising:
an input unit configured to generate operation information based on input by a user, and to send the operation information to the graph processing unit,
wherein a standard value is selected with each item according to the operation information.

7. The ultrasonic diagnostic apparatus according to claim 1, wherein
the graph processing unit is configured to create a graph indicating measuring results of a plurality of fetuses.

8. The ultrasonic diagnostic apparatus according to claim 1, wherein
the graph processing unit is configured to generate a radar chart as the graph.

9. The ultrasonic diagnostic apparatus according to claim 8, wherein
the radar chart includes plural axes, and
the graph processing unit is configured to assign item to each axis of the radar chart.

10. The ultrasonic diagnostic apparatus according to claim 9, wherein
the graph processing unit is configured to normalize a scale of each axis according to standard values, and to assign an average values of measuring results of the assigned items to center parts of respective axes.

11. The ultrasonic diagnostic apparatus according to claim 1, wherein
the graph processing unit is configured to generate a bar chart having a plurality of bars as the graph.

12. The ultrasonic diagnostic apparatus according to claim 11, wherein
the graph processing unit is configured to assign the items to the respective plurality of bars, and create the bar chart so that a particular height of each one of the plurality of bars corresponds to the standard value of the respective item.

13. The ultrasonic diagnostic apparatus according to claim 1, wherein
the graph processing unit is configured to generate a graph indicating measuring results at a plurality of points in time.

14. The ultrasonic diagnostic apparatus according to claim 13, wherein
the graph processing unit is configured to generate a line graph having a first axis indicating time and a second axis indicating relative relation between measuring results and a standard value.

15. A measuring result display apparatus which displays measuring results of fetus according to a plurality of items, comprising:
a graph processing unit configured to generate a graph indicating a relative relation between measuring results and standard values in relation to each of the plurality of items; and
a monitor coupled to the graph processing unit and configured to display the graph.

16. A method of displaying measuring results comprising:
obtaining measuring results of a fetus according to a plurality of items;
generating a graph indicating relative relations between the measuring results and standard values about each of the plurality of items; and
displaying the graph on a monitor.
